(19) United States
(12) Patent Application Publication
(10) Pub. No.: US 2009/0105470 A1
(43) Pub. Date: Apr. 23, 2009

(54) Tin Mediated Regioselective Synthesis of Sucrose-6-Esters

(75) Inventors: Rakesh Ratnam, Mumbai (IN); Mohammed Mofizuddin, Mumbai (IN); Sundeep Aurora, Mumbai (IN)

Correspondence Address: THE NATH LAW GROUP, 112 South West Street, Alexandria, VA, 22314, US

(73) Assignee: PHARMED MEDICARE PRIVATE LIMITED , a corporation

(21) Appl. No.: 11/884,678

(22) PCT Filed: Feb. 6, 2006
(86) PCT No.: PCT/IN2006/000057

§ 371 Date: May 7, 2008

(30) Foreign Application Priority Data
Feb. 22, 2005 (IN) 197/MUM/2005

(51) Int. Cl. C07H 23/00

(52) U.S. Cl. 536/121

(57) ABSTRACT

A method is disclosed for regioselective synthesis of sucrose-6-acetate via formation of a novel sucrose-tin adduct using sucrose and DBTO. The novel tin adduct can be represented by a formula (6-O-sucrose)-O—Snbutyl.sub.2-O-(6-O-sucrose) or as 1,3.(di O-sucrose) dibutyl stannylene. The adduct is acylated to yield sucrose-6-acetate or sucrose-6-benzoate as major product.

### TECHNICAL FIELD

The present invention relates to a process and a novel strategy for synthesis of a sucrose-6-ester, which is a precursor of chlorinated sucrose, 1′-6′-Dichloro-1′-6′-DIDEOXY-β-Fructofuranasyl-4-chloro-4-deoxy-galactopyranoside (TGS). The invention also includes a novel process for synthesis of sucrose-6-esters by a regioselective reaction involving the formation of a novel stannylene intermediate compound.

### BACKGROUND OF INVENTION

Chlorinated sucrose preparation is a challenging process due to the need of chlorination in selective less reactive positions in sucrose molecule in competition with more reactive positions. Generally, this objective is achieved by a procedure which involves essentially protecting the 6-hydroxy group in the pyranose ring of sugar molecule by using various protecting agents alky/aryl anhydride, acid chlorides, orthoesters etc., and the protected sucrose is then chlorinated in the desired positions (1′-6′ &, 4) to give the acetyl derivative of the product, which is then deacylated to give the desired product 1′-6′-Dichloro-1′-6′-DIDEOXY-β-Fructofuranasyl-4-chloro-4-deoxy-galactopyranoside i.e. 4,1′, 6′ trichlorogalactosucrose (TGS).

Strategies of prior art methods of production of TGS described in more details in U.S. Pat. Nos. 4,343,934, 4,362,869, 4,380,476, and 4,435,440 and Indian and International patent applications (563/MUM/2004) WO/2005/090374, WO/2005/090376, 1316/MUM/2004 (PCT/IN05/00408), 1/MUM/2005 (PCT/IN/05/00434), 545/MUM/2005 1047/MUM/2005 1048/MUM/2005 1127/MUM/2005 1173/MUM/2005 1172/MUM/2005 1176/MUM/2005 are analogous to the scope of following process: Sucrose-6-acetate is chlorinated by Vilsmeier-Haack reagent to form 6 acetyl 4,1′, 6′trichlorogalactosucrose (TGS-6-acetate). After chlorination, the deacetylation of 6 acetyl TGS to TGS is carried out in the reaction mixture itself. The TGS is then purified from the reaction mixture in various ways based on selective extraction into water immiscible solvent or solvents. In above reaction, the acetyl group may be any other acyl group too.

The substitution of hydroxyl group in the sucrose molecule by an acyl group is not always occur restricted to the desired 6 position. Generally at normal reaction conditions, esterification occurs at other positions also producing a mixture of sucrose molecules substituted at various positions, which leads to formation of one or more poly-substituted sucrose esters. To isolate the desired sucrose-6-ester from other esters is usually a cumbersome process.

This problem could be overcome only if the reaction would lead to regioselective substitution i.e. substitution at desired position only.

### BRIEF SUMMARY OF THE INVENTION

This invention discloses formation of a novel kind of stannylene adduct as a product of reaction of organo-tin catalyst and sucrose. This invention also discloses a regioselective process for synthesizing sucrose compounds such as 6-substituted sucrose derivatives by improving the chances of occurrence of direction of the reaction specifically to the 6 position only and resulting in preparation of mono-substituted derivatives as a single major product. Preparation of sucrose-6-acetate is but one example to which the invention is applicable. It may find application to more such analogous reactions too.

The process of the invention comprises reacting sucrose with only half mole of DBTO relative to amount of sucrose used to directly produce 1,3.(di O-sucrose) dibutyl stannylene, which is a new/novel adduct.

### THE PRIOR ART

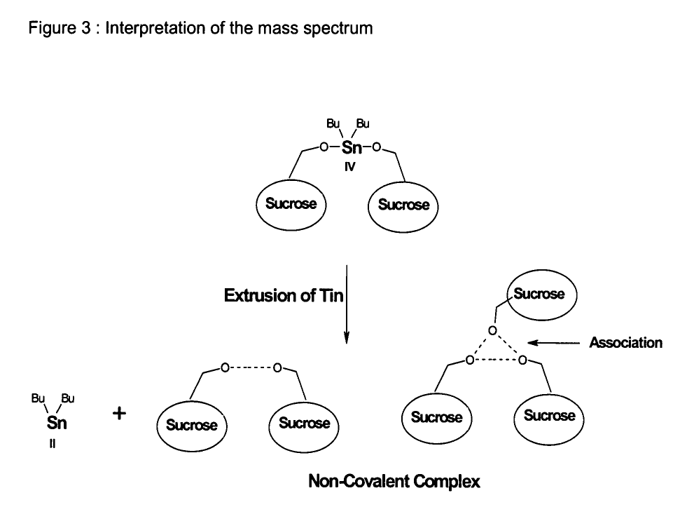

Formation of stannoxyl compounds (FIGS. 3 and 4 in a review article Tetrahedron, Vol 41, No. 4, pp. 643-663, 1985) are disclosed by David et al. as a result of reaction of tin compounds with hydroxyl-group containing compounds such as carbohydrates. Stannoxyl compounds upon alkylation or acetylation shall produce ethers or esters.

Dibutylstannylene derivative of nucleosides is disclosed by Wagner et al., J. Org. Chem., 39, 24 (1974).

Reaction of dibutyltin oxide (DBTO) with 6,1′, 6′-tri-O-tritylsucrose, followed by reaction with benzoyl chloride is reported to have produced a 72% yield of 3′-O-benzoyl-6,1′, 6′-tri-O-tritylsucrose and 9% of the 2-O-benzoate derivative, and minor amounts of the 2,3′-dibenzoate derivative by Holzapfel et al., in “Sucrose Derivatives and the Selective Benzoylation of the Secondary Hydroxyl groups of 6,1′, 6′-tri-O-tritylsucrose”, S. Afr. Tydskr. Chem., 1984, 37(3), pages 57-61.

Navia et al (1990) in U.S. Pat. No. 4,950,746 have disclosed a process which comprises reacting sucrose with a 1,3-di(hydrocarbyloxy)-1,1,3,3-tetra(hydrocarbyl)distannoxane to produce a 1,3-di-(6-O-sucrose)-1,1,3,3 tetra(hydrocarbyl)distannoxane, a new class of compounds, which can then be reacted with an acylating agent to produce a sucrose-6-ester. In a preferred aspect of the invention, the 1,3-di(hydrocarbyloxy)-1,1,3,3-tetra(hydrocarbyl)distannoxane reactant is generated in situ, for example, by reacting a di(hydrocarbyl)tin oxide or equivalent reactant with an alcohol or phenol.

In the method of Navia et al (1990) stoichiometric conversion of the adduct from the reactants, sucrose and DBTO is 1:1 moles respectively to form the distannoxane adduct, the theoretical elemental analysis of which shows tin content of 20.63%

Regioselective methods of substitution using organo-tin catalysts and adducts formed from the same have also been reported by Neiditch et al (1991) in U.S. Pat. No. 5,023,329, Vernon et al (1991) in U.S. Pat. No. 5,034,551, Walkup et al (1992) in U.S. Pat. No. 5,089,608. In none of these is disclosed the adduct of this invention, a process of its preparation and its use in regioselective synthesis of sucrose-6-esters.

### DETAILED DESCRIPTION OF INVENTION

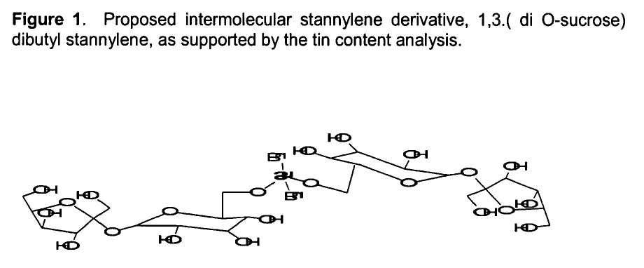

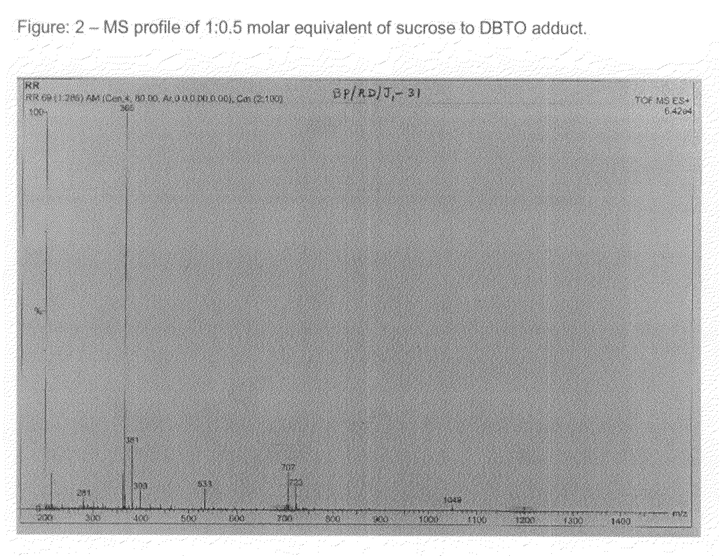

The process of invention comprises of reacting sucrose with dibutyl tin oxide to produce a compound, an adduct which showed around 13.2 to 13.7% tin content and Mass Spectroscopic profile as shown in FIG. 2 which is consistent to the structure of the adduct as 1,3.(di O-sucrose) dibutyl stannylene as shown in FIG. 1. This adduct is a novel adduct, not reported so far. This adduct can then be treated with acylating reagent to form sucrose-6-ester.

In general, the process of this invention comprises dissolving sucrose in N,N-dimethylformamide (DMF) and DBTO is added to it. Instead of DMF, cyclohexane may also be used. Preferred ratio in which sucrose and DBTO are taken for reaction is 1:0.5 molar equivalent of sucrose although 1:1 ratio also gives formation of stannylene of this invention in the same quality and with same composition. The water formed during the reaction needs to be removed continuously. This is achieved in most preferred way when the mixture is heated to 80-85° C. and heating continued for 10-13 hours. DMF is removed, preferably by azeotropic distillation. The adduct is isolated as precipitate from the thick reaction mass by adding methylene chloride, preferably in volume proportion of 1:2.

Although dibutyltin oxide is the organotin catalyst of preferred choice in this invention, the butyl group in the same can be any alkyl, cycloalkyl, aryl or arylalkyl including but not restricted to methyl, ethyl, propyl, butyl, octyl, benzyl, phenethyl, phenyl, naphthyl, cyclohexyl and substituted phenyl. Similarly, instead of oxide, the organotin catalysts can also be a dialkoxide, dihalide, diacylate or another organic tin compound capable of generating a 1,3.(di O-sucrose) di(hydroxycarbyl) stannylene in the reaction mixture analogous in structure to 1,3.(di O-sucrose) dibutyl stannylene.

Preferred solvent for the reaction is DMF or cyclohexane. Basically any alternative solvent that is capable of dissolving sucrose as well as the organotin catalyst chosen (DBTO in the preferred embodiment) may be used. The temperature used for heating and period of its heating are the conditions found economical and convenient. However, any other condition capable of formation of the adduct of this invention, i.e. 1,3.(di O-sucrose) dibutyl stannylene in the preferred embodiment, or any other 1,3 (di O-sucrose) di(hydrocarbyl) stannylene may be used. The adduct of the invention, 1,3 (di O-sucrose) di(hydrocarbyl) stannylene, can also be designated as “di(hydroxycarbyl) stannylene sucrose” represented by following formula:

R′—O—Sn(R)₂—O—R′

wherein each R′ individually represents sucrose-6-ester in the preferred embodiment of this invention, however R′ may also be any other hydroxycarbyl or hydrocarbyl group and wherein each R individually represents a hydrocarbyl group, e.g., alkyl, cycloalkyl, aryl, or aralkyl. Molecules analogous to the adduct of this invention may also include those in which R′ represents alkyl, cycloalkyl, aryl, or aralkyl, and they are also covered within the scope of this invention.

The mechanism of formation of 1,3.(di O-sucrose) dibutyl stannylene, the novel adduct of this invention, is given in FIG. 3. In the prior art process two molecules of the sucrose are said to react with two molecules of tin oxide to give an adduct whereas we have found that the adduct formed from one molecule of tin oxide and two molecules of sucrose after the acylation is enough to afford/explain formation of the desired end product.

The procedures known in the art for separation and purification, including precipitation, crystallization, recrystallization etc. can be used for isolating the di(hydroxycarbyl) stannylene sucrose in addition to the process of precipitation by addition of methylene chloride. The di(hydroxycarbyl) stannylene sucrose may be used further for acylation without further purification, or after purification up to various stages and it may be used in situ i.e. as formed in the reaction mixture without its isolation or after isolation.

The reagent used for acylating the di(hydroxycarbyl) stannylene sucrose is usually around one molar, preferably exceeding a little more but not less than one molar. Preferred acylating reagent is acetic or benzoic anhydride although alternatives capable of acylation may also be used which include, without being limited to acid halides of benzoic and substituted benzoic acid, alkanoic acids, long chain fatty acids, both saturated and unsaturated, unsaturated acids, saturated and unsaturated dicarboxylic, and the like.

Preferred solvent used in this invention for carrying our acylation reaction is N dialkyl substituted amides, most preferred being DMF. However, as long as both reactants and reaction products are soluble in it, same result is achievable by using alternative inert organic solvents or other polar, aproteic compounds.

Range within which reaction worked was seen to be between 75° C. and 100° C. (preferred temperature used here is 80-85° C.) and further heating to 6 to −18 hours (preferred period used being 10-13 hours.

The sucrose-6-ester recovered by using the process of the invention may further be washed free from impurities by using a solvent in which the same is insoluble and impurities are soluble. Acetonitrile or acetone are such solvents useful for a wash.

The advantage of the above said adduct formation and further processing to form sucrose-6-ester is the reduced consumption of the DBTO. It brings down the costing significantly as only 50% of the organotin catalyst is required than the above said process claimed in U.S. Pat. No. 4,950,746.

The working of the invention is illustrated by various examples given below. It shall be reasonably understood that the embodiments and examples described in this specification merely illustrate the invention claimed and do not limit the scope of techniques, reactants, reaction conditions useable which are consistent to the scope of the invention claimed. A modification, an adaptation, a variation of the invention and a product, a process analogous to one which is claimed here which is obvious to the person skilled in the art is also covered within the scope of this disclosure. Similarly a singular also covers pleural unless context does not permit the same. Thus, “a process” includes also “processes” and “a product” also includes “products”.

### EXAMPLE 1

### Preparation of 3(Di O-Sucrose) Dibutyl Stannylene

Sucrose (200 g) was dissolved in 600 ml of DMF and 145.6 g of DBTO and heated to 80-85° C. The heating was continued for 10-13 hrs to remove the water formed during the adduct formation. The reaction mass was cooled and the DMF was removed off completely by azeotropic distillation. The thick mass obtained was treated with 1:2 volumes of methylene chloride.

The solids formed were filtered and washed with excess methylene chloride. The yellow coloured powder obtained was analyzed for tin content.

Same experiment was conducted once again with 200 g of sucrose but with 72.8 g of DBTO in the same manner and the yellow colored powder was analyzed for tin content.

The products were analysed.

### Analytical Methods and Analysis

Tin content analysis: by Atomic absorption spectra
Other elemental analysis: by CHN analyzer
Molecular weight analysis: by GC MS

The mass spectrum of the 1:0.5 molar equivalent of sucrose to DBTO adduct is shown in FIG. 2. In the spectra, the tin is extruded and the two sucrose moieties associate with one more sucrose moiety. The graphical interpretation of mechanism of reaction is illustrated in FIG. 3

The results obtained are given in Table 1

TABLE 1
Tin content of adducts formed at two different relative molar
concentrations of DBTO

| | Tin content as | |
|---|---|---|
| Molar proportion | anticipated theoretically | Tin content as |
| of the reactants | if all the added DBTO | determined by actual |
| for formation | gets incorporated into | chemical analysis of the |
| of an adduct | adduct formation | adduct recovered |
| 1:1 of sucrose: | 20.6% | 13.7% |
| DBTO | | |
| 1:0.5 of sucrose: | 12.9% | 13.2% |
| DBTO | | |

TABLE 2
Elemental analysis of the adducts

| | Composition (%) of the | Composition (%) of the adduct |
|---|---|---|
| | adduct formed (reactants | formed with reactants used in |
| | used in molar proportion of | molar proportion of |
| Element | 1:0.5 sucrose: DBTO) | 1:1 sucrose: DBTO |
| Carbon | 43.2% | 42.62% |
| Hydrogen | 6.54% | 8.35% |
| Oxygen | 34.2% | 29.9% |
| Tin | 13.2% | 13.7% |

The results indicate that the adduct formed by 1:0.5 of sucrose to DBTO is as per the structure provided in the FIG. 1.

The tin content of adducts formed in both the reactions, irrespective of whether the molar ratio of sucrose:DBTO is 1:1 or 1:0.5 show similar tin content indicating that the actual adduct formed in both instances is of same type and structure and mechanism of its formation is also through the same route. The results indicate the structure of the adduct to be as given in FIG. 1.

### EXAMPLE 2

### Sucrose-6-Acetate Synthesis

A) Sucrose-6-Acetate from In Situ Formation of 3.(Di O-Sucrose) Dibutyl Stannylene During the Course of Reaction

Sucrose (200 g) was dissolved in 600 ml of DMF and 72.8 g of DBTO and heated to 80-85° C. The heating was continued for 10-13 hrs to remove the water formed during the adduct formation. Then the reaction mass was cooled to room temperature and chilled to 0° C. 75 ml of acetic anhydride was added dropwise to the reaction mass under stirring. Then the reaction mass was gradually raised to ambient and the Acetylation was monitored by frequent TLC analysis. After about 3-4 hrs, the acetate formation was completed. Then the reaction was terminated by adding 50 ml of water. The DBTO in acetate formed was extracted into 1:2 v/v Cyclohexane twice. Then the layers were separated and the reaction mass is taken for water removal. After azeotropic removal of water is completed, the sucrose-6-acetate was analyzed by HPLC. The results showed 78% conversion of sucrose-6-acetate as the major peak.

Similar experiment was carried out substituting DBTO quantity to 145.6 g and the final conversion obtained was 80% sucrose-6-acetate.

B) Sucrose-6-Acetate from Isolated 3(Di O-Sucrose) Dibutyl Stannylene Formed During the Course of Reaction

500 g of 0.3 (di O-Sucrose) dibutyl Stannylene adduct was dissolved in 500 ml of DMF, heated to 40-45° C. and stirred for 30 minutes for complete dissolution. The reaction mass is then cooled to room temperature and further to 0° C. 75 ml of acetic anhydride was added dropwise to the reaction mass under stirring. Then the reaction mass was gradually raised to ambient and the Acetylation was monitored by frequent TLC analysis. After about 3-4 hrs, the acetate formation was completed. Then the reaction was terminated by adding 50 ml of water. The DBTO in acetate formed was extracted into 1:2 v/v Cyclohexane twice. Then the layers were separated and the reaction mass is taken for water removal. After azeotropic removal of water is completed, the sucrose-6-acetate was analyzed by HPLC. The results showed 79.5% conversion of sucrose-6-acetate as the major peak.

### EXAMPLE 3

### Use of Dioctyltin Oxide for the Conversion of Sucrose to Sucrose-6-Benzoate

Sucrose (20 g) was dissolved in 100 ml of DMF and 10.6 g of Dioctyltin oxide and heated to 85-90° C. The heating was continued for 10-15 hrs to remove the water formed during the adduct formation. Then the reaction mass was cooled to room temperature and chilled to 15° C. Benzoic anhydride 19.8 g (90% pure) was dissolved in 20 ml of DMF and was added dropwise to the reaction mass under stirring. Then the reaction mass was gradually raised to ambient and the benzoylation was monitored by frequent TLC analysis. After about 10-15 hrs, the benzoate formation was completed. Then the reaction was terminated by adding 5 ml of water. The DBTO in benzoate form was extracted into 1:2 v/v Cyclohexane twice. Then the layers were separated and the reaction mass is taken for water removal. After azeotropic removal of water is completed, the sucrose-6-benzoate was analyzed by HPLC. The results showed 85% conversion of sucrose-6-benzoate as the major peak.

### EXAMPLE 4

### Use of Glutaric Anhydride as Acylating Agent to Produce Sucrose-6-Glutarate

Sucrose (10 g) was dissolved in 50 ml of DMF and 3.64 g of DBTO and heated to 80-85° C. The heating was continued for 5-6 hrs to remove the water formed during the adduct formation. Then the reaction mass was cooled to room temperature and chilled to 15° C. 4.3 g of glutaric anhydride was dissolved in 10 ml of DMF and added dropwise to the reaction mass under stirring. Then the reaction mass was gradually raised to ambient and the esterification was monitored by frequent TLC analysis. After about 5-8 hrs, the ester formation was completed. Then the reaction was terminated by adding 3 ml of water. The DBTO in glutarate form was extracted into 1:2 v/v Cyclohexane twice. Then the layers were separated and the reaction mass is taken for water removal. After azeotropic removal of water is completed, the sucrose-6-glutarate was analyzed. The results showed 75% conversion of sucrose-6-glutarate content.

### EXAMPLE 5

### Use of Lauric Anhydride as Acylating Agent to Produce Sucrose-6-Laurate

Sucrose (5 g) was dissolved in 25 ml of DMF and 1.82 g of DBTO and heated to 80-85° C. The heating was continued for 4-5 hrs to remove the water formed during the adduct formation. Then the reaction mass was cooled to room temperature and chilled to 20° C. 7.27 g of Lauric anhydride was dissolved in 15 ml of DMF and added dropwise to the reaction mass under stirring. Then the reaction mass was gradually raised to ambient and the esterification was monitored by frequent TLC analysis. After about 10-15 hrs, the ester formation was completed. Then the reaction was terminated by adding 2 ml of water. The DBTO in laurate form was extracted into 1:2 v/v Cyclohexane twice. Then the layers were separated and the reaction mass is taken for water removal. After azeotropic removal of water is completed, the sucrose-6-laurate was analyzed. The results showed 65% conversion of sucrose-6-laurate content.

### EXAMPLE 6

### Use of Propionic Anhydride as Acylating Agent to Produce Sucrose-6-Propionate

Sucrose (5 g) was dissolved in 25 ml of DMF and 1.82 g of DBTO and heated to 80-85° C. The heating was continued for 4-5 hrs to remove the water formed during the adduct formation. Then the reaction mass was cooled to room temperature and chilled to 20° C. 2.49 g of Propionic anhydride was added dropwise to the reaction mass under stirring. Then the reaction mass was gradually raised to ambient and the esterification was monitored by frequent TLC analysis. After about 3-5 hrs, the ester formation was completed. Then the reaction was terminated by adding 2 ml of water. The DBTO in laurate form was extracted into 1:2 v/v Cyclohexane twice. Then the layers were separated and the reaction mass is taken for water removal. After azeotropic removal of water is completed, the sucrose-6-propionate was analyzed. The results showed 75% conversion of sucrose-6-propionate content.

1. A compound of general formula R′—O—Sn(R)₂—O—R′ wherein each R′ individually represents hydroxycarbyl, alkyl, cycloalkyl, aryl, or aralkyl; and each R individually represents a hydrocarbyl group, e.g., alkyl, cycloalkyl, aryl, or aralkyl.

2. A compound of claim 1 wherein the compound comprises of formula (sucrose-6-ester)-O—Sn(R)₂—O-(sucrose-6-ester), (sucrose-6-ester)-O—Sn(R)₂—O-(sucrose-6-ester), (sucrose-6-ester)-O—Sn(butyl)₂-O-(sucrose-6-ester), (sucrose-6-acetate)-O—Sn(butyl)₂-O-(sucrose-6-acetate), (sucrose-6-benzoate)-O—Sn(butyl)₂-O-(sucrose-6-benzoate), (sucrose-6-glutarate)-O—Sn(butyl)₂-O-(sucrose-6-glutarate), (sucrose-6-laurate)-O—Sn(butyl)₂-O-(sucrose-6-laurate), (sucrose-6-propionate)-O—Sn(butyl)₂-O-(sucrose-6-propionate) and the like.

3. A process of production of compound of claim 1 when the compound is of general formula (sucrose-6-ester)-O—Sn(R)₂—O-(sucrose-6-ester), comprising, sequentially:
a. Dissolving sucrose and a di(hydrocarbyl)tin catalyst, preferably dibutyltin oxide in appropriate molar proportions, preferably around 1:0.5 with respect to moles of sucrose taken, in a solvent, preferably N,N-dimethylformamide (DMF),
b. allowing reaction to occur for a period of time ranging from 6 to 18 hours, preferably for about 10-13 hours under conditions facilitating continuous removal of water, preferably involving continuous heating of the reaction mixture at an elevated temperature preferably at around 80-85 degrees celcius,
c. optionally removing DMF from the reaction mixture by a suitable means including azeotropic distillation,
d. optionally isolating the compound using a method of separation including treatment with methylene chloride in volume sufficient to induce precipitation of the compound of claim 1, preferably with 2 volumes for every volume of the concentrated mass of reactants.

4. A process of production of sucrose-6-ester comprising from a solution comprising a reaction mixture containing compound of general formula (sucrose-6-ester)-O—Sn(R)₂—O-(sucrose-6-ester) comprising the steps of:
a. cooling a solution or a reaction mass to room temperature and chilling it further to about 0° C., if required,
b. acylating the compound of claim 1 by adding, preferably dropwise, under stirring, an acylating reagent preferably in about one molar equivalent quantity of acetic anhydride or benzoic anhydride with respect to the molar concentration of compound of claim 1 in the solution or the reaction mass; or by using an alternative acylating reagent including acid halides of benzoic and substituted benzoic acid, alkanoic acids, long chain fatty acids, both saturated and unsaturated, unsaturated acids, saturated and unsaturated dicarboxylic, and the like,
c. raising the temperature of the reaction mass of sub-claim b. of claim 4 gradually to ambient until completion of acylation in about 3 to 4 hours,
d. terminating the reaction by adding water,
e. extracting the DBTO from the acetate formed into cyclohexane preferably in 1:2 proportion volume/volume, preferably twice,
f. subjecting the reaction mass of sub-claim e. of claim 4 to water removal, preferably by azeotropic distillation.

5. A process of claim 4 wherein sucrose-6-ester comprises sucrose-6-acetate, sucrose-6-benzoate, sucrose-6-glutarate, sucrose-6-laurate, sucrose-6-propionate and like.
